(19) United States
(12) Patent Application Publication
(10) Pub. No.: US 2005/0027195 A1
(43) Pub. Date: Feb. 3, 2005

(54) Calibration data compression

(76) Inventor: Assaf Govari, Haifa (IL)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/633,299

(22) Filed: Aug. 1, 2003

(51) Int. Cl. A61B 5/05

(52) U.S. Cl. 600/433; 600/424

(57) ABSTRACT

A method for calibrating is provided, including receiving, for each of a plurality of frequencies, an indication of a characteristic sensitivity of a position sensor for placement in a patient; measuring an actual sensitivity of the position sensor at each of the plurality of frequencies; and determining, at each of the plurality of frequencies, calibration data indicative of a deviation of the actual sensitivity from the characteristic sensitivity.

### FIELD OF THE INVENTION

The present invention relates generally to systems for medical diagnosis and treatment, and specifically to medical catheters whose location can be detected.

### BACKGROUND OF THE INVENTION

Various methods and devices have been described for determining the position of a probe or catheter tip inside the body using electromagnetic fields, such as in U.S. Pat. No. 5,391,199 to Ben-Haim, European Patent 0 776 176 to Ben-Haim et al. and corresponding US Patent Application Publication 2002/0165448 based on U.S. patent application Ser. No. 09/273,646, U.S. Pat. Nos. 5,833,608 and 6,161,032 to Acker, and U.S. Pat. Nos. 5,558,091 and 5,752,513 to Acker et al., all of which are assigned to the assignee of the present patent application and are incorporated herein by reference. U.S. Pat. No. 5,913,820 to Bladen et al. and U.S. Pat. No. 5,042,486 to Pfeiler et al., both of which are incorporated herein by reference, also describe electromagnetic position-determination systems. Other electromagnetic tracking systems, not necessarily for medical applications, are described in U.S. Pat. Nos. 3,644,825 to Davis, Jr. et al., 3,868,565 and 4,017,858 to Kuipers, 4,054,881 to Raab, and 4,849,692 to Blood, which are likewise incorporated herein by reference.

Because of manufacturing variations, the coils generally used in the position sensors of these position-determining systems to generate position signals may not be precisely oriented with the body of the probe. Additionally, the distance of the coils from the tip of the probe may not be precisely known, and there may be slight variations in the relative gains of the coils in response to externally-applied fields. U.S. Pat. No. 6,266,551 to Osadchy et al., which is assigned to the assignee of the present patent application and is incorporated herein by reference, describes methods and apparatus for pre-calibrating a probe, preferably at the time of manufacture, so as to measure and compensate for variations in the positions, orientations and gains of the coils. To calibrate the probe, a mechanical test fixture holds the probe in one or more predetermined positions and orientations, and radiators generate known, substantially uniform magnetic fields in the vicinity of the test fixture. Signals generated by the coils are analyzed and used to produce calibration data regarding the gains of the coils and deviations of the coils from orthogonality.

Various methods and devices have been described for storing, in a probe, information specific to the probe, such as calibration and identification information. These devices generally include a microchip incorporated in the probe. For example, the above-cited U.S. Pat. No. 6,266,551 to Osadchy et al. describes the incorporation of an electronic microcircuit in a probe, which stores information relating to calibration of the probe. Such information can include an encrypted calibration code and/or a usage code, which controls availability of the probe to a user thereof. Storage of usage codes is also described in US Patent Application Publication 2002/032380 to Acker et al., and U.S. Pat. No. 5,383,874 to Jackson et al., which are incorporated herein by reference. Such usage monitoring codes typically provide an indication representing the number of times the disposable device has been used or the total time the disposable device has been operatively used.

U.S. Pat. No. 6,370,411 to Osadchy et al., which is assigned to the assignee of the present patent application and is incorporated herein by reference, describes a catheter assembly comprising a catheter of minimal complexity and a connection cable which connects the proximal end of the catheter to a console. The catheter comprises a microcircuit which carries substantially only information specific to the catheter, such as calibration data, which is not in common with other catheters of the same model. The calibration data may be recorded in the microcircuit in the catheter in the form of lookup tables, polynomial coefficients or any other suitable form known in the art. The cable comprises an access circuit which receives the information from the catheter and passes it in a suitable form to the console.

U.S. Pat. No. 6,248,083 to Smith et al., which is incorporated herein by reference, describes a guide wire assembly having a measuring device mounted in the distal end portion thereof. It also has an interface cable which includes information storage, containing calibration/temperature compensation data, uniquely characteristic of the measuring device. The calibration data are used with uncompensated output from the measuring device to calculate a correct measurement value.

U.S. Pat. No. 5,617,857 to Chader et al., which is incorporated herein by reference, describes an imaging system which determines the location of a medical instrument. A read-only storage device is positioned on or in the medical instrument for storing initialization information characteristic of the instrument.

PCT Patent Publication WO 00/33755 to Tierney et al., which is incorporated herein by reference, describes robotic surgical tools, which include a memory. Among the memory functions is an indication of tool-specific information including measured calibration offsets, indicating misalignment of the tool drive system, tool life data, or the like.

### SUMMARY OF THE INVENTION

In embodiments of the present invention, a catheter system comprises a catheter and a memory device, which is typically incorporated into the catheter. The catheter further comprises a position sensing device, which typically comprises one or more coils, such as three substantially orthogonal non-concentric coils. In order to compensate for manufacturing variations, each of the coils is calibrated, generally at the time of manufacture, by measuring the gains of the coils in response to known magnetic fields at one or more frequencies. The deviations of these measured gains from expected values, rather than the measured gains themselves, are stored in the memory device. Because such deviations are generally small and fall within a narrow range of values, storage of such deviations rather than the measured gains generally reduces consumption of the limited memory typically available in the memory device. For some applications, a test fixture is used to calibrate the gains of the coils, such as by utilizing techniques described in the above-cited U.S. Pat. No. 6,266,551 to Osadchy et al.

The memory device is typically incorporated in a microcircuit, and generally comprises an EEPROM, EPROM, PROM, Flash ROM, or non-volatile RAM. Because of space limitations inherent in catheter design, small memory components having limited data capacity are typically used to store the calibration data, as mentioned above. For example, one appropriately-sized EPROM has a total memory of 512 bits. However, it is often desirable to store a relatively large amount of calibration data. For some applications, the same coil is used with externally-applied fields of different frequencies, each frequency having unique calibration data. In addition, when more than one coil is incorporated in the catheter, calibration data from the multiple coils is typically stored in the same memory component. Furthermore, the same memory component is sometimes used to store additional calibration data, such as data relating to the positions and orientations of the coils in the catheter. The use of the calibration storage techniques described herein generally allows the storage of more calibration data and/or the use of smaller memory devices than would otherwise be possible.

There is therefore provided, in accordance with an embodiment of the present invention, a method for calibrating, including:
receiving, for each of a plurality of frequencies, an indication of a characteristic sensitivity of a position sensor for placement in a patient;
measuring an actual sensitivity of the position sensor at each of the plurality of frequencies; and
determining, at each of the plurality of frequencies, calibration data indicative of a deviation of the actual sensitivity from the characteristic sensitivity.

For some applications, determining the calibration data at each of the plurality of frequencies includes calculating by subtraction a difference between the actual sensitivity and the characteristic sensitivity. For some applications, determining, at each of the plurality of frequencies, the calibration data indicative of the deviation includes expressing the deviation as a proportion of the characteristic sensitivity. For some applications, determining, at each of the plurality of frequencies, the calibration data indicative of the deviation includes representing the deviation in a non-linear manner with respect to the plurality of frequencies.

In an embodiment, the position sensor includes a plurality of coils, and determining the calibration data, at each of the plurality of frequencies, includes determining the calibration data for each of the plurality of coils.

In an embodiment, the position sensor includes at least one coil, and determining the calibration data, at each of the plurality of frequencies, includes determining the calibration data responsive to an actual gain and a characteristic gain of the coil.

In an embodiment, the position sensor includes at least one coil, and determining the calibration data, at each of the plurality of frequencies, includes determining the calibration data responsive to at least one of: a position of the coil within the position sensor and an orientation of the coil within the position sensor.

In an embodiment, the position sensor is incorporated in a device for placement within the patient, and determining the calibration data, at each of the plurality of frequencies, includes determining the calibration data responsive to at least one of: a position of the position sensor within the device and an orientation of the position sensor within the device.

For some applications, the method includes storing the calibration data in the position sensor.

There is also provided, in accordance with an embodiment of the present invention, a method for determining a position, including:
placing a position sensor in a patient;
generating one or more fields at one or more respective frequencies;
generating one or more position signals, responsive to the respective fields and a position and an orientation of the position sensor;
retrieving, for at least one of the one or more frequencies, a stored value of a deviation of an actual sensitivity of the position sensor from a characteristic sensitivity of the position sensor;
determining, for the at least one of the one or more frequencies, a correction to the respective position signal, responsive to the respective position signal and the value of the deviation; and
determining the position of the position sensor, responsive to the one or more position signals and the correction.

For some applications, determining the correction, for the at least one of the one or more frequencies, includes adding the value of the deviation to the respective position signal. For some applications, the deviation is expressed as a proportion of the characteristic sensitivity, and determining the correction, for the at least one of the one or more frequencies, includes determining the correction responsive to the respective position signal and the proportion.

For some applications, the deviation is represented in a non-linear manner with respect to the one or more frequencies, and determining the correction, for the at least one of the one or more frequencies, includes determining the correction responsive to the respective position signal and the value of the deviation represented in the non-linear manner.

There is further provided, in accordance with an embodiment of the present invention, apparatus for calibrating a position sensor for placement in a patient, the apparatus including:
a test fixture, adapted to hold the position sensor in a known position and orientation;
a plurality of radiator coils, adapted to generate fields at a plurality of frequencies; and
a computer, adapted to:
receive, for each of the plurality of frequencies, an indication of a characteristic sensitivity of the position sensor,
measure an actual sensitivity of the position sensor, responsive to the fields generated at each of the plurality of frequencies, and
determine, at each of the plurality of frequencies, calibration data indicative of a deviation of the actual sensitivity from the characteristic sensitivity.

There is still further provided, in accordance with an embodiment of the present invention, apparatus including a device adapted to be placed into a patient, the device including:
a position sensor; and
a memory, which stores calibration data indicative of a deviation, at each of a plurality of frequencies, of an actual sensitivity of the position sensor from a characteristic sensitivity of the position sensor.

In an embodiment, the device is adapted to be incorporated in an elongate probe. Alternatively, the device is adapted to be incorporated in a capsule, adapted to be placed in the patient.

For some applications, the deviation includes a difference between the actual sensitivity and the characteristic sensitivity, determined using subtraction, and the memory is adapted to store the calibration data indicative of the difference. For some applications, the deviation is expressed as a proportion of the characteristic sensitivity, and the memory is adapted to store the calibration data indicative of the proportion. For some applications, the deviation is represented in a non-linear manner with respect to the plurality of frequencies, and the memory is adapted to store the calibration data indicative of the non-linear representation of the deviation.

In an embodiment, the position sensor includes at least one coil. For some applications, the at least one coil includes a plurality of coils, and the memory is adapted to store, at each of the plurality of frequencies, the calibration data for each of the plurality of coils.

For some applications, the actual sensitivity of the position sensor is indicative of an actual gain of the coil, the characteristic sensitivity of the position sensor is indicative of a characteristic gain of the coil, and the memory is adapted to store the calibration data indicative of a deviation, at each of the plurality of frequencies, of the actual gain from the characteristic gain.

For some applications, the calibration data is indicative of at least one of: a position of the coil within the position sensor and an orientation of the coil within the position sensor. For some applications, the calibration data is indicative of at least one of: a position of the position sensor within the device and an orientation of the position sensor within the device.

There is yet further provided, in accordance with an embodiment of the present invention, apparatus for position determination, including:
a plurality of radiator coils, adapted to generate fields at one or more frequencies;
a device, adapted to be placed into a patient, the device including:
   a position sensor; and
   a memory, adapted to store calibration data indicative of a deviation, at each of a plurality of frequencies, of an actual sensitivity of the position sensor from a characteristic sensitivity of the position sensor,
   the position sensor adapted to generate one or more position signals responsive to the respective fields and a position and an orientation of the position sensor; and
circuitry, adapted to:
receive the position signals, and
determine the position of the position sensor, responsive to the position signals and the calibration data.

The present invention will be more fully understood from the following detailed description of embodiments thereof, taken together with the drawings in which:

### BRIEF DESCRIPTION OF THE DRAWINGS

FIG. 1 is a simplified pictorial illustration of a system including a catheter, in accordance with an embodiment of the present invention;

FIG. 2 is a schematic, pictorial illustration of the distal end of the catheter of FIG. 1, in accordance with an embodiment of the present invention;

FIG. 3 is a detailed schematic view of a connector at the proximal end of the catheter, in accordance with an embodiment of the present invention;

FIG. 4 is a graph of sensitivity vs. frequency for a coil in the catheter of FIG. 1, in accordance with an embodiment of the present invention;

FIG. 5 is a flow chart that schematically illustrates a method for manipulating and storing calibration data of a coil of the catheter of FIG. 1, in accordance with an embodiment of the present invention; and

FIG. 6 is a schematic, pictorial illustration of an implantable or insertable encapsulated transponder 600, in accordance with an embodiment of the present invention.

### DETAILED DESCRIPTION OF EMBODIMENTS

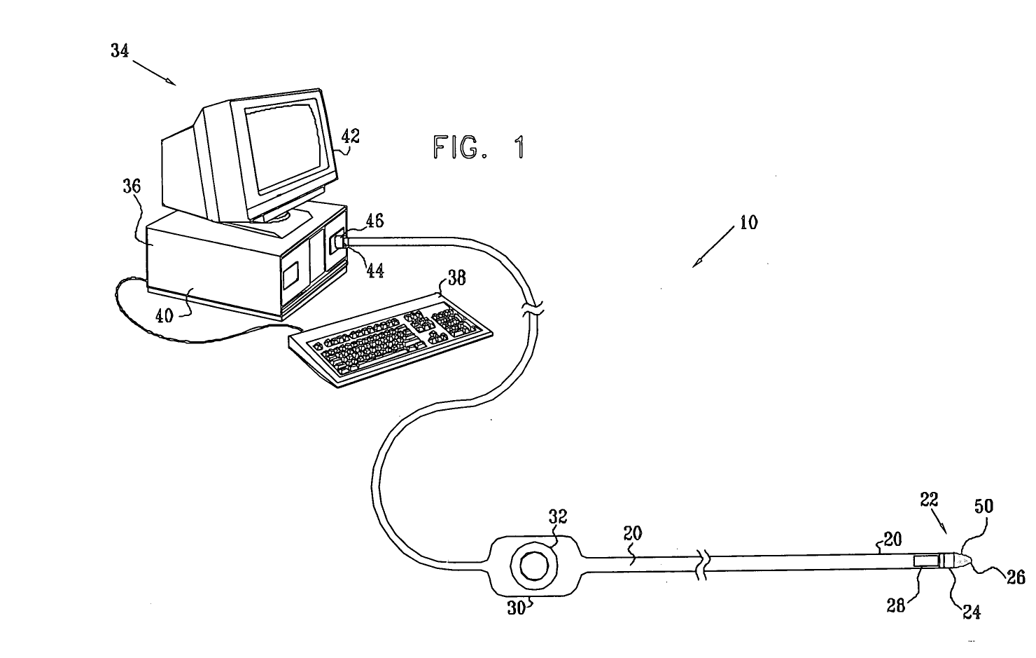

FIG. 1 is a simplified pictorial illustration of a catheter system 10 comprising an elongate probe, such as a catheter 20, for insertion into the human body, in accordance with an embodiment of the present invention. It is to be understood that although the following embodiments are described with reference to a catheter, embodiments of the present invention are equally applicable to use with other types of probes.

Catheter 20 typically includes a handle 30 for operation of the catheter by a user. Controls 32 on handle 30 enable the user to steer a distal end 22 of the catheter in a desired direction, or to position and/or orient distal end 22 as desired.

System 10 further comprises a console 34, which enables the user to observe and regulate the functions of catheter 20. Console 34 typically includes a computer 36, a keyboard 38, signal processing circuits 40, which are typically inside the computer, and a display 42. Signal processing circuits 40 typically receive, amplify, filter, and digitize signals from catheter 20, whereupon these digitized signals are received and used by computer 36 to compute the position and orientation of the catheter. Catheter 20 is typically coupled at its proximal end by a connector 44 to a mating receptacle 46 on console 34.

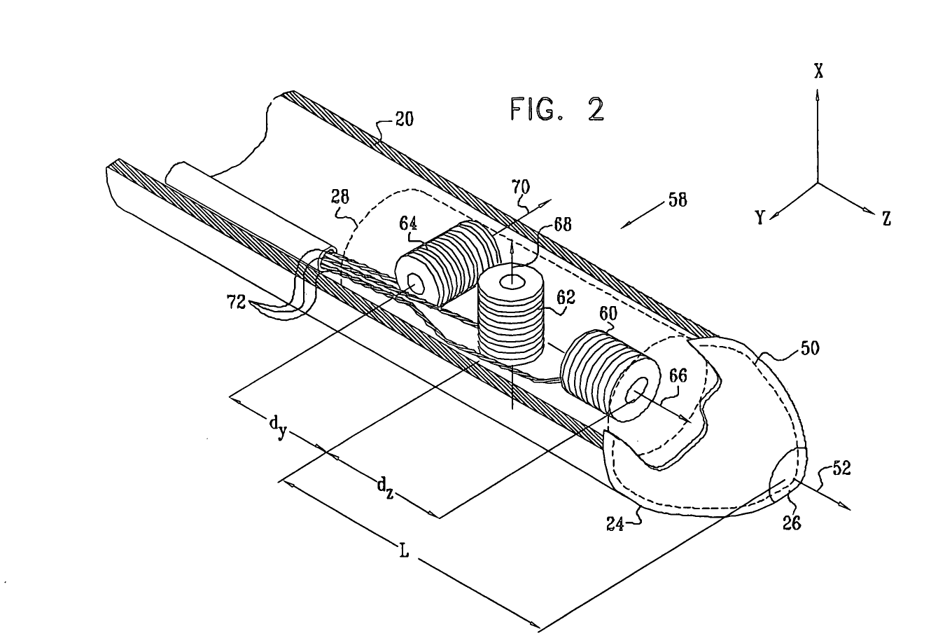

FIG. 2 is a schematic, pictorial illustration of distal end 22 of catheter 20, in accordance with an embodiment of the present invention. Distal end 22 comprises a functional portion 24 for performing diagnostic and/or therapeutic functions, adjacent to a distal tip 26 of the catheter. Functional portion 24 typically comprises a tool 50, such as an ultrasound transducer or ablation electrode. Distal end 22 of catheter 20 further comprises a position sensing device 28 that generates signals used to determine the position and orientation of the catheter within the body. Position sensing device 28 is typically adjacent to functional portion 24. There is typically a fixed positional and orientational relationship between position sensing device 28 and portion 24.

Position sensing device 28 typically comprises one or more coils 58, e.g., three non-concentric coils 60, 62 and 64, such as described in the above-cited European Patent 0 776 176 and U.S. patent application Ser. No. 09/273,646 to Ben-Haim et al. This device enables continuous generation of six dimensions of position and orientation information. Coils 60, 62 and 64 have respective axes 66, 68 and 70 which typically, but not necessarily, define orthogonal Cartesian axes Z, X and Y, respectively, as shown in FIG. 2, wherein the Z-axis is parallel to the long axis of catheter 20 and the X- and Y-axes define a plane perpendicular thereto. The coils each have a fixed position and orientation with respect to each other.

Although embodiments of the present invention are described herein with reference to the position signal generating device shown in FIG. 2 and described above, it is to be understood that the inventive concepts of the present invention are similarly applicable to probes including other position sensing devices. For example, embodiments of the present invention may comprise a single coil for generating position signals, or two or more such coils, which may be concentric or non-concentric. Other embodiments of the present invention may comprise other types of position sensing devices, such as Hall effect devices.

As shown in FIG. 2, position sensing device 28 is located in catheter 20 at a distance L from distal tip 26, where L is defined herein for convenience as the distance along the Z-axis from central axis 68 of coil 62 to tip 26. Respective axes 66 and 70 of coils 60 and 64 are displaced from axis 68 by respective distances dy and dz.

Signal processing circuits 40 in console 34 receive signals carried by coil wires 72 from coils 60, 62, and 64, and convey them to computer 36, which computes the three-dimensional translational position of position sensing device 28 and the rotational orientation of axes 66, 68 and 70, relative to a fixed, external coordinate frame. The actual position and orientation of distal tip 26 are then computed by taking into account the distance L of tip 26 from the center of position sensing device 28, as defined by axis 68, and the orientation of axes 66, 68 and 70.

It has been found empirically that because of deviations in the process of manufacturing catheter 20 and coils 58, variations in the respective gains of the coils may cause errors in determination of position and orientation of the catheter. In addition, the distance L typically varies from one catheter to another, leading to errors in calculating the position of tip 26. Furthermore, axis 66 of coil 60 typically deviates from absolute alignment with the long axis of catheter 20, which passes through tip 26. Moreover, axes 68 and 70 of coils 62 and 64 respectively are typically not precisely orthogonal to axis 66 or to each other, thereby inducing additional errors in determination of position and orientation of the catheter. Still furthermore, there are typically variations in the distances dy and dz.

Therefore, in an embodiment of the present invention, position sensing device 28 is calibrated, taking into account variations in the respective gains of the coils, and, optionally, one or more of the other variations described above, before the catheter is inserted into a patient's body. In an embodiment, this calibration is performed using one or more test fixtures (not shown) and methods of calibration described in the above-mentioned U.S. Pat. No. 6,266,551 to Osadchy et al. For example, the '551 patent describes a jig comprising three mutually orthogonal pairs of parallel radiator coils mounted on a base. Each radiator coil pair generates a predetermined, substantially uniform magnetic field that is substantially normal to the planes defined by the pair of coils, and is thus substantially orthogonal to fields generated by the other two radiator coil pairs. In order to calibrate the gains of the coils, total amplitudes of the respective catheter coil signals are derived by summing the squares of the amplitudes of the signals generated by each of the catheter coils in response to each of the radiator coil pairs in turn. Since the magnetic fields in the vicinity of coils 60, 62 and 64 have equal and substantially uniform components along each of the coil axes 66, 68 and 70, the total signal amplitudes will be independent of the respective orientations and positions of coils 60, 62 and 64, and will depend only on the respective coil gains.

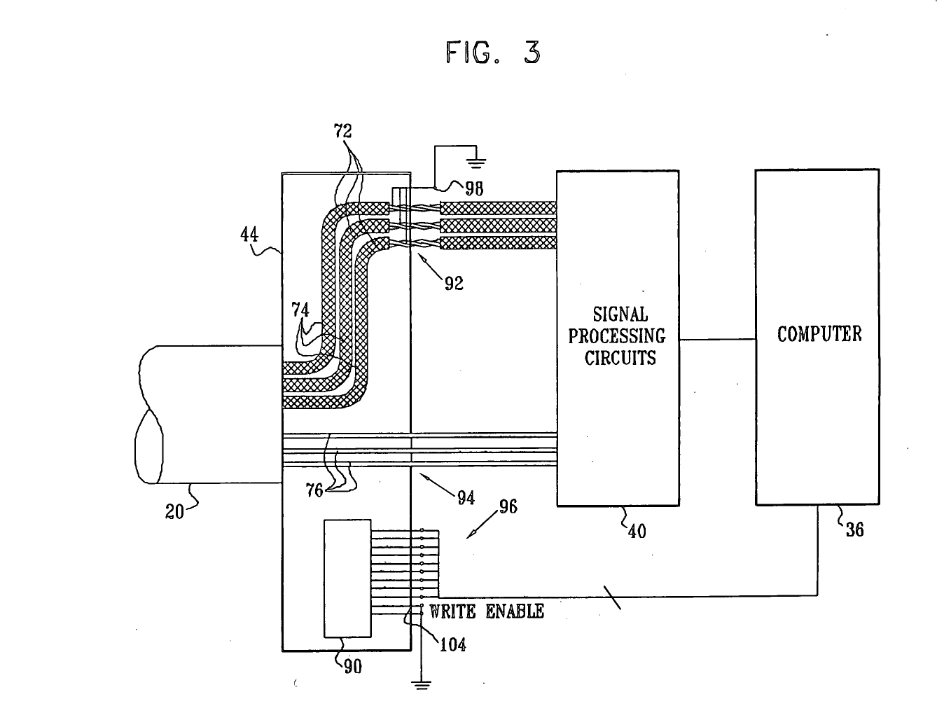

Reference is now made to FIG. 3, which is a detailed schematic view of connector 44 at the proximal end of catheter 20, in accordance with an embodiment of the present invention. For some applications, connector 44 comprises a digital microcircuit 90, comprising a memory 91 in which calibration data for catheter 20 are electronically stored. Memory 91 comprises an EEPROM, an EPROM, a PROM, a Flash ROM, non-volatile RAM, or other type of programmable memory known in the art. Alternatively, microcircuit 90 is located elsewhere in catheter 20, rather than in connector 44. For example, the microcircuit may be located in a vicinity of handle 30.

In the embodiment shown in FIG. 3, connector 44 comprises pins 92, 94, 96, and 98, which mate with corresponding sockets in receptacle 46 on console 34 (FIG. 1). Typically, functional pins 94 carry analog electrophysiological signals conveyed over functional wires 76 to signal processing circuits 40. Coil pins 92 typically carry position and orientation signals conveyed by coil wires 72 from coils 58 to signal processing circuits 40 and computer 36, which computes the position and orientation of catheter 20. The computer further reads digital calibration correction function data stored in microcircuit 90 via memory pins 96, and uses these data in computing the correct catheter position and orientation. In embodiments in which microcircuit 90 is located elsewhere in catheter 20, the microcircuit is typically coupled to memory pins 96 over a set of wires (not shown).

For some applications, one or more write-enable pins 104 are likewise coupled to microcircuit 90. These pins are used to enable programming of the microcircuit with the desired calibration data. At the time of calibration, the write-enable input is enabled, and calibration data are recorded in the microcircuit. Thereafter the write-enable input is disabled, for example by removing the write-enable pin or by connecting it to electrical ground, as shown in FIG. 3, so that further calibration data may not be recorded in the microcircuit, and the microcircuit functions in a read-only mode.

Alternatively, in embodiments of the present invention wherein microcircuit 90 comprises an EEPROM device, the write-enable input may be disabled by sending a write-protect command to the device. This command may be reversible or irreversible.

In an embodiment of the present invention, microcircuit 90 comprises a device incorporating password-secured access control, and write-access to the microcircuit requires that an appropriate password first be entered. The microcircuit is programmed with calibration data at the time of manufacture, and thereafter operates in a “read access only” mode, with all write operations locked out, or in a “read access and program only” mode, in which certain data, but not calibration data, may be written to the device. Changing the mode of operation of the microcircuit requires that an appropriate password be entered, which password is generally unavailable to users of the system.

In an embodiment of the present invention, microcircuit 90 comprises an EPROM or PROM device, which is contained in the catheter connector, and the input and output connections of the EPROM or PROM are coupled to pins of the connector. Calibration data are recorded in the EPROM or PROM at the time of manufacture using a suitable programming device, not shown in the figures, which receives data from the computer used in calibration. The programming device is connected to catheter connector 44 and programs the EPROM or PROM by inputting digital signals thereto through the connector. Thereafter, the EPROM or PROM may not be re-programmed.

In some embodiments of the present invention, data recorded in microcircuit 90 include a calibration code, which is encrypted in accordance with methods known in the art, so as to ensure that the calibration data have not been altered or corrupted. The calibration code typically includes a checksum. When the user connects catheter 20 to console 34, computer 36 reads the calibration code and compares the code with pre-programmed values. If the code does not match the desired pre-programmed value, the computer causes a message to be displayed by display 42 indicating that the catheter may not be appropriately calibrated. The computer may further cause the system to cease operation until a catheter having a code matching the desired pre-programmed value is connected thereto.

Typically, the calibration code is encrypted using a method that prevents decryption by unauthorized parties, for example the RSA encryption scheme, using a public key and a private key, or other methods known in the art. When a method such as RSA encryption is used, the private key is known only to authorized manufacturers of the catheter, so as to prevent the possible use of unauthorized substitutes of possibly inferior quality.

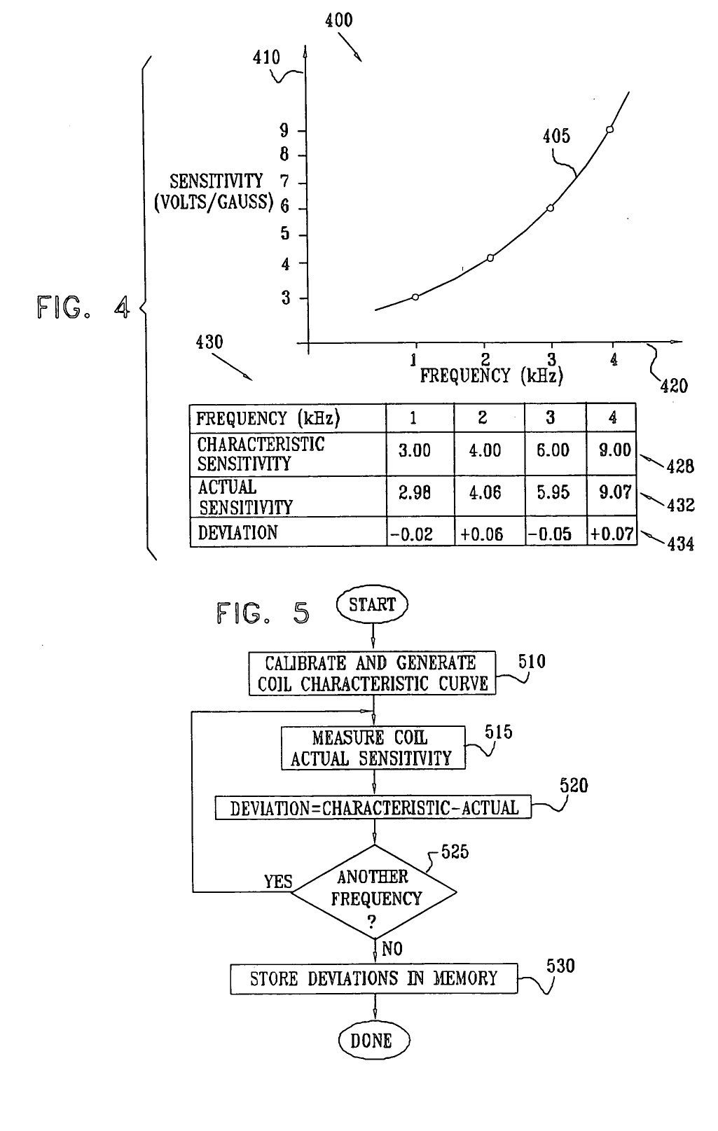

Reference is now made to FIG. 4, which is a graph 400 of sensitivity vs. frequency for one of coils 58, in accordance with an embodiment of the present invention. Each type of coil has a characteristic curve 405, which represents the sensitivity of the coil at different field frequencies. A horizontal axis 420 of graph 400 indicates frequency (typically expressed in kHz), while a vertical axis 410 of the graph represents the sensitivity of the coil (typically expressed in volts/gauss) at each frequency. The sensitivity values are typically calculated based on the design of the coil, or empirically determined for a large number of frequencies. For example, in the characteristic curve shown in FIG. 4, the coil exhibits a calculated sensitivity of 3.00 volts/gauss at a frequency of 1 kHz, and of 6.00 volts/gauss at 3 kHz. Depending on the type of coil, the characteristic curve may be non-linear, as shown in FIG. 4, or linear.

In practice, because of manufacturing variances, as described hereinabove, a specific coil 58 often has sensitivities at each frequency that differ from the values of characteristic curve 405. A row 428 of a table 430 shows exemplary characteristic sensitivity values, i.e., expected values, at several frequencies (indicated immediately above the table on axis 420), while a second row 432 of the table shows exemplary actual measured sensitivity values for the coil. For example, at 1 kHz, the actual sensitivity, as determined during a calibration procedure, is 2.98 volts/gauss, which is 0.02 volts/gauss less than the calculated sensitivity of 3.00 volts/gauss. For each frequency represented in table 430, the deviation between characteristic sensitivity and actual sensitivity is indicated in a row 434. The coil is calibrated for each frequency for which it is desirable to store calibration data. Optionally, such calibration is performed using procedures described in the above-mentioned U.S. Pat. No. 6,266,551 to Osadchy et al.

FIG. 5 is a flow chart that schematically illustrates a method for manipulating and storing calibration data of one of coils 58 in memory 91, in accordance with an embodiment of the present invention. A characteristic curve, representing theoretical sensitivities of ideal coils of the same type as coil 58, is generated, as described hereinabove, at a characteristic curve generation step 510. As part of a typical manufacturing process, one or more coils 58 are then installed in position sensing device 28 and the actual sensitivity of each of the coils is measured at a chosen frequency, at an actual sensitivity measurement step 515, as described hereinabove. Alternatively, the actual sensitivity of each coil is measured prior to installation of the coil in position sensing device 28. A deviation is calculated for each coil, typically by subtracting the actual sensitivity from the characteristic sensitivity, at a deviation calculation step 520. If there are additional frequencies at which it is desired to calibrate coils 58, as determined at an additional frequency check step 525, the method returns to step 515.

On the other hand, if the coils have been calibrated at all desired frequencies, respective deviations are stored in memory 91, as described hereinbelow, at a store deviation step 530. For some applications, additional calibration data are stored at step 530, e.g., data relating to the position and/or orientation of one or more coils in the catheter, and/or data relating to the position and/or orientation of tool 50.

At step 530, the calculated deviations, rather than the actual sensitivities, are stored in memory 91, thereby reducing the memory consumption. Based on the range of acceptable manufacturing tolerances, a predetermined number of bits of memory is typically allocated in memory 91 for each frequency. For example, if the deviation values have a range of +0.08 to −0.07 volts/gauss, sixteen possible deviation values (including 0.00, representing no deviation) can be stored in four bits (a nibble) of data, for a single coil at a single frequency. Alternatively, a non-linear representation of the deviations is used, in which, for example, values closer to 0.00 are allotted more bits, thereby providing greater precision in the representation of these values. Further alternatively, each deviation is expressed as a percentage value of the characteristic sensitivity or other proportion of the characteristic sensitivity, and an appropriate number of bits is selected to represent the deviation. Such a percentage calculation may be appropriate, for example, in applications in which absolute deviation values are generally greater at higher frequencies, but the deviations, when expressed as a percentage of the characteristic sensitivity, are generally constant across the frequencies. Other methods of calculating the deviation will be readily apparent to those skilled in the art, having read the present patent application, and are considered within the scope of the present invention.

During use of the probe during a procedure, the amplitudes of signals received from these coils are adjusted using the calibration data stored in memory 91.

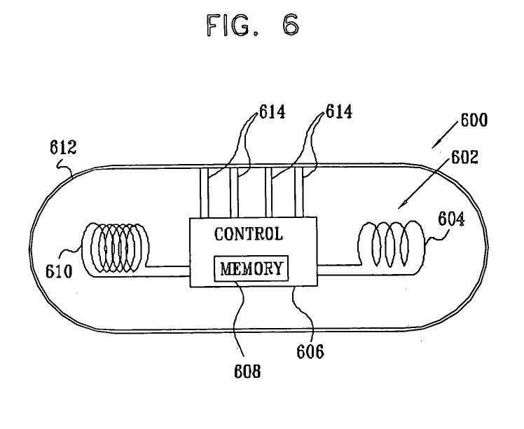

Reference is now made to FIG. 6, which is a schematic, pictorial illustration of an implantable or insertable encapsulated transponder 600, in accordance with an embodiment of the present invention. Transponder 600 comprises a position sensing device 602 that generates signals used to determine the position and orientation of the transponder within the body. Position sensing device 602 is typically similar to position sensing device 28, and typically comprises one or more sensing coils 604 similar to coils 58. Transponder 600 further comprises a control chip 606, which is typically similar to control chip 44. Control chip 606 comprises a memory 608, generally similar to memory 91. Transponder 600 typically additionally comprises a coil 610, for receiving externally-generated power and transmitting position information, using analog or digital signals. Transponder 600 may utilize techniques described in U.S. patent application Ser. No. 10/029,473 to Govari, which is assigned to the assignee of the present application and is incorporated herein by reference. (Position sensing device 28, coils 58, control chip 44, and memory 91 are described hereinabove with reference to FIG. 2.)

Transponder 600 is typically encapsulated in sealed packaging 612, such as ceramic packaging. For some applications, control chip 606 comprises electronic contact pins 614, which enable the control chip to be programmed from an external device. Typically, sealed packaging 612 covers the pins, which are only used for initialization of transponder 600 during manufacture and calibration prior to sealing.

In an embodiment of the present invention, position sensing device 602 is calibrated before transponder 600 is inserted into a patient's body, using the calibration and data storage techniques described hereinabove, mutatis mutandis. For some applications, the position sensing device is calibrated prior to application of sealed packaging 612, in which case pins 614 are typically used to transmit the calibration data for storage in memory 608. Alternatively, the calibration is performed after transponder 600 has been sealed, and the calibration data are transmitted wirelessly to coil 610 or coil 604 for storage in memory 608.

It will thus be appreciated that the embodiments described above are cited by way of example, and that the present invention is not limited to what has been particularly shown and described hereinabove. Rather, the scope of the present invention includes both combinations and subcombinations of the various features described hereinabove, as well as variations and modifications thereof which would occur to persons skilled in the art upon reading the foregoing description and which are not disclosed in the prior art.

1. A method for calibrating, comprising:
receiving, for each of a plurality of frequencies, an indication of a characteristic sensitivity of a position sensor for placement in a patient;
measuring an actual sensitivity of the position sensor at each of the plurality of frequencies; and
determining, at each of the plurality of frequencies, calibration data indicative of a deviation of the actual sensitivity from the characteristic sensitivity.

2. The method according to claim 1, wherein determining the calibration data at each of the plurality of frequencies comprises calculating by subtraction a difference between the actual sensitivity and the characteristic sensitivity.

3. The method according to claim 1, wherein determining, at each of the plurality of frequencies, the calibration data indicative of the deviation comprises expressing the deviation as a proportion of the characteristic sensitivity.

4. The method according to claim 1, wherein determining, at each of the plurality of frequencies, the calibration data indicative of the deviation comprises representing the deviation in a non-linear manner with respect to the plurality of frequencies.

5. The method according to claim 1, wherein the position sensor includes a plurality of coils, and wherein determining the calibration data, at each of the plurality of frequencies, comprises determining the calibration data for each of the plurality of coils.

6. The method according to claim 1, wherein the position sensor includes at least one coil, and wherein determining the calibration data, at each of the plurality of frequencies, comprises determining the calibration data responsive to an actual gain and a characteristic gain of the coil.

7. The method according to claim 1, wherein the position sensor includes at least one coil, and wherein determining the calibration data, at each of the plurality of frequencies, comprises determining the calibration data responsive to at least one of: a position of the coil within the position sensor and an orientation of the coil within the position sensor.

8. The method according to claim 1, wherein the position sensor is incorporated in a device for placement within the patient, and wherein determining the calibration data, at each of the plurality of frequencies, comprises determining the calibration data responsive to at least one of: a position of the position sensor within the device and an orientation of the position sensor within the device.

9. The method according to claim 1, comprising storing the calibration data in the position sensor.

10. A method for determining a position, comprising:
placing a position sensor in a patient;
generating one or more fields at one or more respective frequencies;
generating one or more position signals, responsive to the respective fields and a position and an orientation of the position sensor;
retrieving, for at least one of the one or more frequencies, a stored value of a deviation of an actual sensitivity of the position sensor from a characteristic sensitivity of the position sensor;
determining, for the at least one of the one or more frequencies, a correction to the respective position signal, responsive to the respective position signal and the value of the deviation; and
determining the position of the position sensor, responsive to the one or more position signals and the correction.

11. The method according to claim 10, wherein determining the correction, for the at least one of the one or more frequencies, comprises adding the value of the deviation to the respective position signal.

12. The method according to claim 10, wherein the deviation is expressed as a proportion of the characteristic sensitivity, and wherein determining the correction, for the at least one of the one or more frequencies, comprises determining the correction responsive to the respective position signal and the proportion.

13. The method according to claim 10, wherein the deviation is represented in a non-linear manner with respect to the one or more frequencies, and wherein determining the correction, for the at least one of the one or more frequencies, comprises determining the correction responsive to the respective position signal and the value of the deviation represented in the non-linear manner.

14. Apparatus for calibrating a position sensor for placement in a patient, the apparatus comprising:
a test fixture, adapted to hold the position sensor in a known position and orientation;
a plurality of radiator coils, adapted to generate fields at a plurality of frequencies; and
a computer, adapted to:
receive, for each of the plurality of frequencies, an indication of a characteristic sensitivity of the position sensor,
measure an actual sensitivity of the position sensor, responsive to the fields generated at each of the plurality of frequencies, and
determine, at each of the plurality of frequencies, calibration data indicative of a deviation of the actual sensitivity from the characteristic sensitivity.

15. The apparatus according to claim 14, wherein the computer is adapted to determine the calibration data, at each of the plurality of frequencies, by calculating by subtraction a difference between the actual sensitivity and the characteristic sensitivity.

16. The apparatus according to claim 14, wherein the computer is adapted to determine, at each of the plurality of frequencies, the calibration data indicative of the deviation by expressing the deviation as a proportion of the characteristic sensitivity.

17. The apparatus according to claim 14, wherein the computer is adapted to determine, at each of the plurality of frequencies, the calibration data indicative of the deviation by representing the deviation in a non-linear manner with respect to the plurality of frequencies.

18. The apparatus according to claim 14, wherein the position sensor includes a plurality of coils, and wherein the computer is adapted to determine, at each of the plurality of frequencies, the calibration data for each of the plurality of coils.

19. The apparatus according to claim 14, wherein the position sensor includes at least one coil, and wherein the computer is adapted to determine the calibration data, at each of the plurality of frequencies, responsive to an actual gain and a characteristic gain of the coil.

20. The apparatus according to claim 14, wherein the position sensor includes at least one coil, and wherein the computer is adapted to determine the calibration data, at each of the plurality of frequencies, responsive to at least one of: a position and of the coil within the position sensor an orientation of the coil within the position sensor.

21. The apparatus according to claim 14, wherein the position sensor is incorporated in a device for placement in the patient, and wherein the computer is adapted to determine the calibration data, at each of the plurality of frequencies, responsive to at least one of: a position of the position sensor within the device and an orientation of the position sensor within the device.

22. The apparatus according to claim 14, wherein the computer is adapted to store the calibration data in the position sensor.

23. Apparatus comprising a device adapted to be placed into a patient, the device comprising:
a position sensor; and
a memory, which stores calibration data indicative of a deviation, at each of a plurality of frequencies, of an actual sensitivity of the position sensor from a characteristic sensitivity of the position sensor.

24. The apparatus according to claim 23, wherein the device is adapted to be incorporated in an elongate probe.

25. The apparatus according to claim 23, wherein the device is adapted to be incorporated in a capsule, adapted to be placed in the patient.

26. The apparatus according to claim 23, wherein the deviation includes a difference between the actual sensitivity and the characteristic sensitivity, determined using subtraction, and wherein the memory is adapted to store the calibration data indicative of the difference.

27. The apparatus according to claim 23, wherein the deviation is expressed as a proportion of the characteristic sensitivity, and wherein the memory is adapted to store the calibration data indicative of the proportion.

28. The apparatus according to claim 23, wherein the deviation is represented in a non-linear manner with respect to the plurality of frequencies, and wherein the memory is adapted to store the calibration data indicative of the non-linear representation of the deviation.

29. The apparatus according to claim 23, wherein the position sensor comprises at least one coil.

30. The apparatus according to claim 29, wherein the at least one coil comprises a plurality of coils, and wherein the memory is adapted to store, at each of the plurality of frequencies, the calibration data for each of the plurality of coils.

31. The apparatus according to claim 29,
wherein the actual sensitivity of the position sensor is indicative of an actual gain of the coil,
wherein the characteristic sensitivity of the position sensor is indicative of a characteristic gain of the coil, and
wherein the memory is adapted to store the calibration data indicative of a deviation, at each of the plurality of frequencies, of the actual gain from the characteristic gain.

32. The apparatus according to claim 29, wherein the calibration data is indicative of at least one of: a position of the coil within the position sensor and an orientation of the coil within the position sensor.

33. The apparatus according to claim 29, wherein the calibration data is indicative of at least one of: a position of the position sensor within the device and an orientation of the position sensor within the device.

34. Apparatus for position determination, comprising:
a plurality of radiator coils, adapted to generate fields at one or more frequencies;
a device, adapted to be placed into a patient, the device comprising:
a position sensor; and
a memory, adapted to store calibration data indicative of a deviation, at each of a plurality of frequencies, of an actual sensitivity of the position sensor from a characteristic sensitivity of the position sensor,
the position sensor adapted to generate one or more position signals responsive to the respective fields and a position and an orientation of the position sensor; and
circuitry, adapted to:
receive the position signals, and
determine the position of the position sensor, responsive to the position signals and the calibration data.

35. The apparatus according to claim 34, wherein the deviation includes a difference between the actual sensitivity and the characteristic sensitivity, determined using subtraction, and wherein the memory is adapted to store the calibration data indicative of the difference.

36. The apparatus according to claim 34, wherein the deviation is expressed as a proportion of the characteristic sensitivity, and wherein the memory is adapted to store the calibration data indicative of the proportion.

37. The apparatus according to claim 34, wherein the deviation is represented in a non-linear manner with respect to the plurality of frequencies, and wherein the memory is adapted to store the calibration data indicative of the non-linear representation of the deviation.
